(19) United States
(12) Patent Application Publication
(10) Pub. No.: US 2009/0043335 A1
(43) Pub. Date: Feb. 12, 2009

(54) Nontraumatic Two-Point Needle for Surgical Suture

(76) Inventor: Sergio Capurro, Genova (IT)

Correspondence Address: COLEMAN SUDOL SAPONE, P.C., 714 COLORADO AVENUE, BRIDGE PORT, CT, 06605-1601, US

(21) Appl. No.: 11/887,863

(22) PCT Filed: May 17, 2006
(86) PCT No.: PCT/EP2006/004638

§ 371 Date: Oct. 4, 2007

(30) Foreign Application Priority Data
May 18, 2005 (IT) GE2005 A000033

(51) Int. Cl. A61B 17/06

(52) U.S. Cl. 606/222; 606/223

(57) ABSTRACT

A nontraumatic two-point needle for surgical suture having two or more modular components constituted by hollow and solid needle portions to be assembled by locking the suture line, or lines, inside the hollow portion or portions.

The present invention relates to a nontraumatic two-point needle for surgical suture.

Prior art nontraumatic two-point needles are constituted by a monolithic body with a solid or hollow structure and by a line tying system which is based on inserting and locking the line by introducing it within a slot or hole provided in the wall of the needle.

The main drawback of the prior art needles is that threading the two-point needle is a long and awkward process. If the needle is solid, tying the end of the line in a slot is not reliable. If instead the two-point needle is hollow, inserting the line in a non-through hole provided in the wall is difficult, and so is the subsequent system for tying the line inside the needle.

Another disadvantage is that the non-through hole provided in the wall of the needle is necessarily larger than the suture line and constitutes a possible receptacle of material during surgical maneuvers.

Another drawback of such needles is that the rim of the hole, on which the suture line rubs during the operation, may possibly wear and eventually cut the suture line.

Also, the prior art needles do not allow to use more than one suture line simultaneously, which would be very useful in some special surgical applications.

The aim of the present invention is to overcome the cited prior art problems.

A particular object of the invention is to provide a needle which allows to easily and rapidly tie a suture line to the needle.

A further particular object of the invention is to provide a needle that allows to tie more than one suture line simultaneously.

The above aims and other aims that will be more apparent hereinafter, are achieved by a nontraumatic two-point needle for surgical suture.

Further characteristics and advantages of the invention will be more apparent by the following description of embodiments of the invention, illustrated, by way of example in the enclosed drawings wherein:

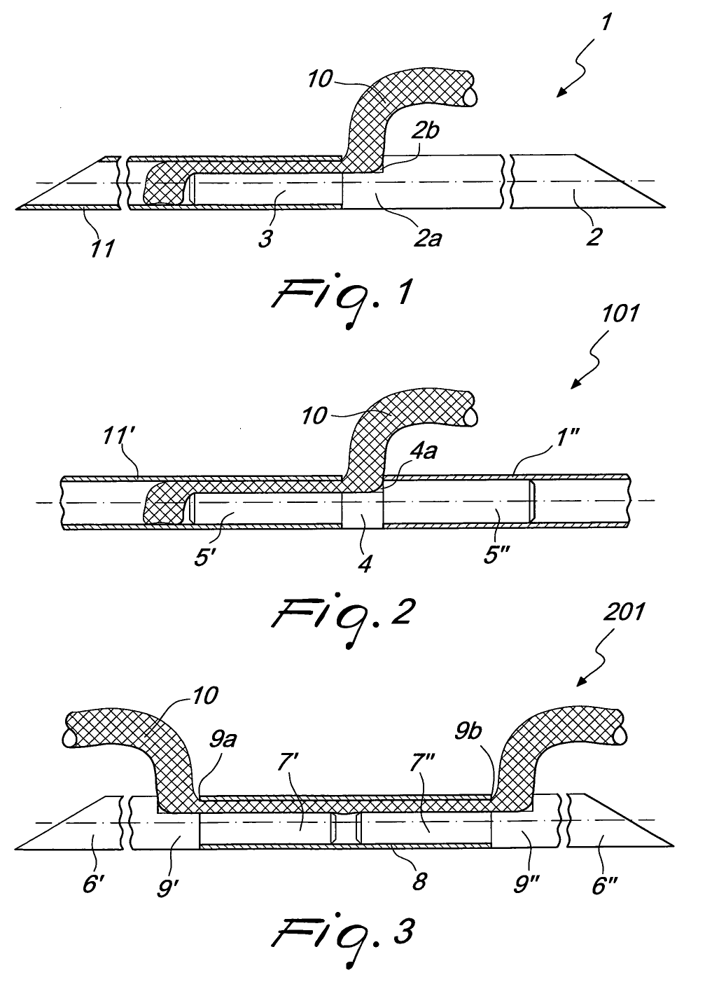

FIG. 1 is a side sectional view of an embodiment of the needle according to a first aspect of the invention;

FIG. 2 is a view similar to the previous one but illustrating a needle according to a further aspect of the invention;

FIG. 3 is a view similar to the previous one but illustrating a needle according to a further aspect of the invention;

In the various figures, similar components have been designated by the same reference numerals.

FIG. 1 illustrates a two-point needle 1 constituted by two portions: a first hollow portion 11, in which the tip at one of the two ends is rounded and/or sharp or variously shaped, and a second solid portion 2 which has, in transverse cross-section, the same dimension as the hollow portion 11.

The second portion 2 has a point at one end and a smaller segment 3, at the other end.

When the needle 1 is assembled, the smaller segment 3 is snugly inserted within the hollow portion 11 but has a longitudinal groove that forms a gap with the inner surface of the hollow portion 11. Such gap is adapted to contain a portion of a suture line 10.

The needle 1 is threaded by inserting a terminal portion of the suture line 10 and then the smaller segment 3 of the solid portion 2, in the hollow portion 11, at the opposite end with respect to the tip.

As shown in FIG. 1, between the solid portion 2 and the smaller segment 3 there is a portion 2a, which is provided externally with the same profile as the hollow portion 11 and the solid portion 2, but in which there is a seat 2b which accommodates the suture line 10.

FIG. 2 illustrates a two-point needle 101 constituted by three portions: two hollow terminal portions 11′ and 11″, with the point at one of the two ends which is rounded and/or sharp or variously shaped, and a third solid intermediate portion 4, having two smaller segments 5′ and 5″ at its two ends.

The solid portion 4 substantially has the same profile of the hollow portions 11′ and 11″ except that a seat 4a is provided in order to accommodate the suture line 10.

When the needle 101 is assembled, the smaller segments 5′ and 5″ are snugly inserted in the hollow portions 11′ and 11″ but one of the segments, segment 5′ in the illustrated embodiment, has a longitudinal groove that forms a gap with the inner surface of the hollow portion 11′. Such gap is adapted to contain a portion of a suture line 10.

The needle 101 is threaded by inserting a terminal portion of the suture line 10 and the smaller segment 5′ of the solid portion 4 in the hollow portion 11′, at the opposite end with respect to the point.

Subsequently the other smaller segment 5″ of the solid portion 4 is inserted in the other hollow portion 11″ again at the opposite end with respect to the point.

According to a further aspect of the invention, also the smaller segment 5″ has a longitudinal groove that forms a gap with the inner surface of the other hollow portion 11″. Such gap is adapted to contain a portion of a second suture line 10. In such manner the needle 101 may be connected to two suture lines.

FIG. 3 illustrates a two-point needle 201 constituted by three portions: two solid end portions 6′ and 6″, with the point at one of the two ends which is rounded and/or sharp or variously shaped, and a smaller segment 7′ and 7″ at the other end, and a third hollow intermediate portion 8.

Between the solid portions 6′ and 6″ and the smaller segments 7′ and 7″, there is a portion 9′, 9″, which has the same external profile as the solid portions 6′ and 6″, but has seats 9a and 9b which are suitable to accommodate the suture line 10.

The needle 201 is threaded by inserting the line 10 and then the smaller segment 7′ of the solid portion 6′, at one of the two ends of the hollow portion 8. The smaller segment 7″ of the other solid portion 6″ is inserted at the other end of the hollow portion 8, optionally also preceded by a suture line. The suture line can pass through the hollow portion 8.

In all of the illustrated embodiments, the opening defined by the seat, 2b, 4a, 9a or 9b, is variable, adjusting it to fit the size of the suture line.

These embodiments, even in their multi-modular versions, are particularly advantageous: the insertion of the intermediate portions can in fact be repeated several times, so as to be able to use multiple suture lines simultaneously.

In other words, the solid or hollow intermediate portions 3, 5′, 5″, 8 can be assembled repeatedly together with their complementary hollow or solid portions 1, 1′, 1″, 7′, 7″.

By producing hollow portions 1, 1′, 1″, 8 and solid portions 2, 4, 6′, 6″ of various lengths, it is possible to assemble longer or shorter needles according to the requirements.

In all the types of embodiment, in the assembly step, for greater safety, the smaller segment that enters the hollow portion can be glued and/or crimped and/or inserted by pressing and/or welded.

The suture line is tied during the assembly step, and the smaller segment has a shape which consolidates the structure and facilitates the locking of the thread: for example, it can have a section which lacks a segment so that an empty part that can accommodate the thread remains inside the needle, or it can have another geometrical shape with a slot which allows to fix a larger thread.

The needle can be straight and/or curved and/or angled.

A nontraumatic needle with two sharp or rounded points for surgical suture has been provided, constituted by two, three or more modular components, and adapted to tie one or more surgical lines.

When assembled, the needle according to the invention provides a precise and suitable opening for the exit of the suture line, and constitutes the two-point needle.

One or both ends, depending on the embodiments, of the solid portion have a reduced size, so that they can be inserted in the hollow portion and can consolidate the structure, with the possibility to allow the fixing of one or more threads.

Some of the many advantages achieved with the present invention are as follows.

The suture line is inserted easily in the hollow portion and its tying is easy and quick. The suture line in fact no longer needs to be inserted in a hole provided in the wall, but is inserted straight in the end of the hollow portion, where it is fixed during the assembly of the needle.

The assembly system allows to rapidly tie multiple suture lines to the same needle, which is composed of multiple hollow and solid portions that are mutually assembled in a modular manner.

In such manner, two or more offset surgical lines may be inserted with a single path of the needle; such offset lines ensure traction in different areas in the same region of the body.

A further advantage of the invention is constituted by the fact that the opening for the surgical line can be perfectly adapted to the diameter of the line itself which is therefore less subject to small movements and friction that, in the prior art needles, entailed the possible tearing of the line on the rim of the hole, with severe consequences.

A further advantage of the present invention is the possibility to change the length of the needle by changing the length of just one of the portions that constitute it.

The needle according to the invention may have numerous modifications and variations, within the scope of the appended claims. All the details may be substituted with technically equivalent elements and the materials employed, as well as the dimensions, may be any according to the specific needs and the state of the art.

1. A nontraumatic two-point needle for surgical suture, comprising two or more modular components.

2. The two-point needle according to claim 1, wherein the modular components are constituted by hollow and solid needle portions to be assembled, consolidating the needle.

3. The two-point needle according to claim 2, wherein one or both ends of the solid portion have a segment of reduced size or smaller segment, which is inserted in the hollow portion, in which a surgical line is inserted beforehand.

4. The two-point needle according to claim 2, further comprising two portions: a first hollow portion with the point at one of the two ends which is rounded and/or sharp or variously shaped and accommodates said line inside it, and a second solid portion, having a transverse cross-section of substantially the same dimensions as the hollow portion, with a point at one end which is rounded and/or sharp or variously shaped, and a smaller segment at the other end, which is suitable to be inserted within the hollow portion.

5. The two-point needle according to claim 2, also comprising three portions: two hollow end portions with the tip at one of the two ends which is rounded and/or sharp or variously shaped, and a solid intermediate portion with two smaller segments at the two ends, which are inserted within said hollow portions.

6. The two-point needle according to claim 2, additionally comprising three portions, a hollow intermediate portion and two solid end portions, with the point at one of the two ends which is rounded and/or sharp or variously shaped, and a smaller segment at the other end, said end portions being inserted in said hollow portion.

7. The two-point needle according to claim 4, wherein between the solid portion and the smaller segment there is a portion which externally has the same profile as the hollow portion and as the solid portion, but in which there is a seat which accommodates said line that exits from the needle.

8. The two-point needle according to claim 5, wherein the solid portion has, on part of the outside perimeter, the profile of the hollow portion, but has a seat formed therein which accommodates said line that exits from the needle.

9. The two-point needle according to claim 6, wherein between the solid portions and the smaller segments there is a portion which has the same outside profile as the solid portions but has seats which accommodates said line that exits from the needle.

10. The two-point needle according to claim 2, wherein one or more non-through lines are inserted in the hollow portion or portions.

11. The two-point needle according to claim 2, wherein one or more through lines are inserted in the hollow portion or portions.

12. The two-point needle according to claim 3, wherein the smaller segment or segments that enter the hollow portion are glued and/or crimped and/or inserted by pressing and/or welded.

13. The two-point needle according to claim 3, wherein one or more parts are removed in the smaller segment or segments in order to allow the passage of one or more lines.

14. The two-point needle according to claim 3, wherein the smaller segment or segments are provided with one or more slots which allow the passage and tying of said line.

15. The two-point needle according to claim 3, wherein the smaller segment that is not used for the passage of said line has a solid cross-section, without removed portions or slots.

16. The two-point needle according to claim 1, wherein it can be straight and/or curved and/or angled.

17. The two-point needle according to claim 2, wherein the solid or hollow intermediate portions are assembled repeatedly with their hollow or solid complementary portions in a modular manner, constituting a needle from which two or more lines exit.
